(19) United States
(12) Patent Application Publication
(10) Pub. No.: US 2025/0367650 A1
(43) Pub. Date: Dec. 4, 2025

(54) IRIDIUM-BASED CATALYST AND PREPARATION METHOD THEREFOR, AND METHOD FOR CATALYZING PROPYLENE HYDROFORMYLATION

(71) Applicant: NANJING INSTITUTE OF MICROINTERFACE TECHNOLOGY CO., LTD, Nanjing, Jiangsu (CN)

(72) Inventors: Xingbang HU, Nanjing, Jiangsu (CN); Chenfei YAO, Nanjing, Jiangsu (CN); Zhibing ZHANG, Nanjing, Jiangsu (CN); Zheng ZHOU, Nanjing, Jiangsu (CN); Lei LI

(21) Appl. No.: 18/872,800

(22) PCT Filed: Feb. 13, 2023
(86) PCT No.: PCT/CN2023/075586

§ 371 Date: Dec. 6, 2024

(30) Foreign Application Priority Data
Feb. 13, 2023 (CN) 202310102750.9

(51) Int. Cl. B01J 31/24; B01J 37/04; B01J 37/08

(52) U.S. Cl. CPC B01J 31/2404; B01J 37/04; B01J 37/08; B01J 2231/321; B01J 2531/827

(57) ABSTRACT

Provided in the present invention are an iridium-based catalyst and a preparation method therefor, and a method for catalyzing propylene hydroformylation. The structural formula of the iridium-based catalyst is (I), wherein Ph is phenyl, x is any one of 0, 1 and 2, and X is any one of Cl, NO₃, BF₄, PF₆ and acac. The iridium-based catalyst disclosed in the present invention has a good catalytic effect and high activity and can still maintain a relatively high catalytic performance after repeated use.

### FIELD OF THE INVENTION

The present invention relates to the technical field of hydroformylation synthesis, and in particular to an iridium-based catalyst and preparation method therefor, and a method for catalyzing propylene hydroformylation.

### BACKGROUND OF THE INVENTION

Aldehydes have always been an important chemical product with a wide range of uses, which can be further used in the synthesis of pesticides, plasticizers, detergents and other high value-added products. The global demand for aldehyde production capacity is up to millions of tonnes per year, and the hydroformylation of olefins and syngas to prepare aldehydes is attracting attention due to its efficient atom economy and high selectivity for aldehydes.

So far, the most commonly reported catalysts for catalyzing the hydroformylation of olefins are mainly rhodium-based catalysts. For example, triphenylphosphine-rhodium is used as a catalyst in patents WO00200583, EP3712126A1, CN102826967 and CN114401940A. Sodium triphenyphosphine trisulfonate-rhodium is used as a catalyst in the patent CN107737609A; trisilylphosphine-rhodium is used as a catalyst in the patent CN111333680B; bidentate dialkylphosphine-rhodium is used as the catalyst in the patent CN113754615A; bidentate phosphite-rhodium is used as a catalyst in the patent CN106000470B; tetradentate phosphine ligand-rhodium with a complex structure, is used as a catalyst in the patent CN115124572A.

In addition to the above patents, there is a large number of literatures that also optimize and improve ligands. For example, Journal of Organometallic Chemistry. 2002, 654, 83-90 uses polyether phosphite as ligand, and Molecular Catalysis. 2017, 434, 116-122 uses triphenylphosphine derivatives: phosphine ligands with large steric hindrance are used to improve the catalytic performance of rhodium-based catalysts. Angew. Chem. Int. Ed. 2019, 58, 2120-2124 employed a chiral phosphine ligand; and Appl. Organometal. Chem. 2013, 27, 313-317 uses diphosphorimide as a ligand.

However, in addition to triphenylphosphine ligands, the other ligands mentioned above not only have complex synthetic routes, but also have high synthesis costs, which will undoubtedly increase production costs. In addition, due to the high activity of traditional rhodium-based catalysts to catalyze the hydroformylation of olefins, attentions are focused on the optimization of rhodium-based catalysts in recent years. However, the price of rhodium is extremely expensive and not suitable for mass production.

In view of this, the present invention is proposed.

### SUMMARY OF THE INVENTION

A first objective of the present invention is to provide a reaction system for preparing soda ash. The reaction system combines micro-interface technology into the soda ash preparation process, effectively improves the reaction efficiency of raw materials and the utilization rate of carbon dioxide, and simultaneously reduces the input pressure of carbon dioxide and reduces energy consumption.

A first objective of the present invention is to provide an iridium-based catalyst, the catalyst has good activities, low cost, and remarkable catalytic effects. Under the premise that tripyridyl phosphine is used as a ligand, the performance of the iridium-based catalyst is better than that of the rhodium-based catalyst, and the price of the iridium metal is one-fourth of the price of rhodium metal. In practical industrial applications, catalyst costs are more saving.

A second objective of the present invention is to provide a preparation method for the above-mentioned iridium-based catalyst, which is simple in steps and can quickly prepare an iridium-based catalyst with excellent catalytic effect in massive quantities.

A third objective of the present invention is to provide a method for catalyzing propylene hydroformylation by using the above-mentioned iridium-based catalyst. The process of the method is simple, the conditions are mild, and the adopted iridium-based catalyst is low-priced, which can save production cost, and realize industrial production.

In order to achieve the above purpose, the present invention adopts the following technical scheme:

The present invention provides an iridium-based catalyst, wherein a structural formula of the iridium-based catalyst is:+

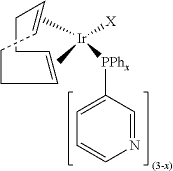

wherein, Ph is phenyl, x is any one of 0, 1, 2, and X is any one of Cl, NO₃, BF₄, PF₆ and acac; wherein, Cl is chloride ion, NO₃ is nitrate, BF₄ is tetrafluoroborate, PF₆ is hexafluorophosphate, and acac is any one of acetylacetone roots.

The invention also provides a preparation method of the above-mentioned iridium-based catalyst, which comprises the following steps: under the protection of inert gas, adding dropwise a tetrahydrofuran solution of phosphine ligand to a tetrahydrofuran solution of iridium compound, stirring after dropping, and vacuum evaporating the tetrahydrofuran solution at 25-30° C. to obtain a target product.

For iridium-based catalysts, extra attention needs to be paid to the temperature of vacuum evaporation in the preparation process, and the temperature limit of vacuum evaporation must be guaranteed to be between 25-30° C., which is due to the reason that when producing iridium-based catalysts, evaporation at high temperature will cause the activity of the produced catalyst to decrease, and metal clusters may also be generated, resulting in the inactivation of the produced catalysts. At the same time, because the structure of the iridium-based catalyst produced by the present invention is sensitive to temperature, the catalyst is easily inactivated when the temperature is high. Therefore, in order to ensure that the produced iridium-based catalyst has good activity and excellent catalytic effect, it is necessary to limit the temperature of vacuum evaporation. At the same time, reducing the temperature can also prevent the phosphine ligand from being oxidized during the reaction process and ensure the stability of the iridium-based catalyst. Therefore, in order to ensure that the above-mentioned iridium-based catalyst with a specific structure can be obtained, the temperature of vacuum evaporation must be limited.

Preferably, a temperature of dropwise addition is 0-30° C., a temperature of stirring is 20-40° C., and the inert gas is one of argon or nitrogen.

Preferably, the temperature of dropwise addition is 5° C., the temperature of stirring is 30° C., and the temperature of vacuum evaporation is 30° C.

Preferably, a molar ratio of the iridium compound to the phosphine ligand is 1:2; and the iridium compound is iridium chloride cyclooctadiene.

Preferably, the phosphine ligand is any one or more of tripyridyl-phosphine, dipyridyl-phenyl phenyl-phosphine, and pyridyl-diphenyl-phosphine.

Based on the status quo described in the background art, the objective of the present invention is to develop a new type of catalyst that is cheaper than rhodium metal, and the catalytic effect is comparable to that of rhodium-based catalyst at the same time. Because iridium metal and rhodium metal are homogeneous elements, so it is thought that iridium metal has a similar catalytic effect. However, the factor that determines the catalytic effect is not only the metal element itself, but also includes a ligand that plays a significant role. After many experiments, the ligands given above are the three with the best effect. Therefore, the invention discloses the above-mentioned iridium-based catalyst to solve the status quo in the background art.

The invention also provides a hydroformylation reaction method for catalyzing propylene using a catalyst, which includes the following steps:
adding the catalyst and a corresponding solvent, dispersing evenly, and adding propylene, carbon monoxide and hydrogen for reaction;
wherein the catalyst is selected from the above-mentioned iridium-based catalyst;
wherein the catalyst accounts for 0.01-1.0 wt % of a solvent mass;
preferably, the catalyst accounts for 0.2 wt % of the solvent mass.

In order to further understand the reaction steps of the catalytic reaction, the following figure is given:

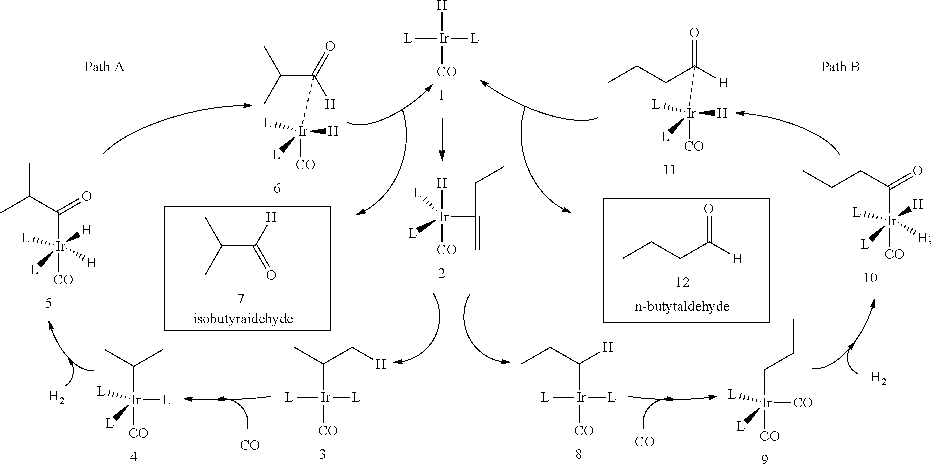

As can be seen from the above accompanying figure, during the reaction of the iridium-based catalyst, the produced iridium-based catalyst (1) is composed of the central atom metal iridium and two phosphine ligands, which is a catalytic form with a vacancy coordination. The propylene then coordinates with the central atom metal iridium to form an intermediate (2). At this point, there are two possible reaction paths: 1) When Path A is followed, the intermediate (2) is directly bonded to the central atom metal iridium after inserting a β-carbon atom into the propylene, forming an intermediate (>C—Ir, 3); CO is then inserted and coordinated with the central atom metal iridium to form an intermediate (4); Hydrogen oxidation is added to the intermediate (4) to form an intermediate (5); finally, the reductive and elimination reaction forms a complex of a ligand catalyst (6) and a branched hydroformylation product (isobutyl aldehyde, 7). At the same time, the iridium-based catalyst is reduced to its original catalytic form (1), thus completing the catalytic cycle. 2) The basic process of Path B is similar to that of Path A, the difference is that when propylene is inserted into the intermediate (2), the α-carbon atom at the end of the propylene is directly bonded to the central atom metal iridium to form an intermediate ('C—Ir, 8), and then a linear hydroformylation product (n-butyraldehyde, 12) is generated through a process similar to Path A. In the figure above, L represents the phosphine ligand.

Preferably, a partial pressure ratio of the carbon monoxide to the hydrogen is 1: (0.2-5).

Preferably, the partial pressure ratio of the carbon monoxide to the hydrogen is 1:1.

Preferably, the solvent is a combination of any one or more of n-butyraldehyde, isobutyraldehyde, toluene, acetonitrile, N-methyl-pyrrolidone, 1,4-dioxane, N,N-dimethylformamide.

Preferably, the solvent is acetonitrile.

Preferably, a temperature of the reaction is 80-120° C., and a pressure of the reaction is 1.5-3.8 MPa.

Preferably, the temperature of the reaction is 100° C., and the pressure of the reaction is 1.9 MPa.

Preferably, an addition of iridium metal in the iridium-based catalyst relative to the solvent is 0.003-0.3 wt %.

Preferably, the addition of iridium metal in the iridium-based catalyst relative to the solvent is 0.07 wt %.

Compared with the prior art, the present invention has at least the following advantages:
(1) The iridium-based catalyst provided by the present invention takes tripyridyl-phosphine as a ligand, wherein its catalytic effect is much higher than that of rhodium-based catalyst, and the price of iridium metal is one-fourth of the price of rhodium metal. In practical application, a large amount of production cost can be saved.
(2) The iridium-based catalyst provided by the present invention is simple in preparation, the conditions are mild, and the energy consumption is low. The reaction activity is obviously better than that of the rhodium-based catalyst under the same reaction conditions.
(3) The present invention adopts an iridium-based catalyst as a catalyst, and propylene, carbon monoxide and hydrogen are used as reaction gases to conduct hydroformylation reaction. Because the price of iridium metal is low, the cost is greatly reduced. Because the adopted catalyst is an iridium-phosphine catalyst, the reaction conditions of the catalytic process are mild, and the positive and heterogeneous ratio of aldehydes is adjustable.

### BRIEF DESCRIPTION OF THE DRAWINGS

Various other advantages and benefits will become clear to those of ordinary skill in the art upon reading the following detailed description of the preferred embodiments. The drawings are for the purpose of illustrating preferred embodiments only and are not to be construed as limiting the present invention. Also, throughout the drawings, the same reference signs are used to appoint the same components. In the drawings:

FIG. 1 is the chromatogram of tripyridyl-phosphine iridium-based catalyst for catalyzing propylene hydroformylation provided in Embodiment 1 of the present invention;

FIG. 2 is the chromatogram of dipyridyl-phenyl-phenyl-phosphine iridium-based catalyst for catalyzing propylene hydroformylation provided in Embodiment 3 of the present invention;

FIG. 3 is the chromatogram of pyridine-diphenyl-phosphine iridium-based catalyst for catalyzing propylene hydroformylation provided in Embodiment 4 of the present invention.

### DESCRIPTION OF THE PREFERRED EMBODIMENTS

The schemes of the present invention will be clearly and completely described below in conjunction with the accompanying drawings and specific embodiments. However, those skilled in the art will understand that the following described embodiments are some of the embodiments of the present invention, rather than all of them. They are only used to illustrate the present invention and should not be regarded as limiting the scope of the present invention. Based on the embodiments of the present invention, all other embodiments obtained by those of ordinary skill in the art without creative efforts fall within the scope of the protection of the present invention. If the specific conditions are not specified in the embodiments, the conditions should be conducted according to the conventional conditions or the conditions recommended by the manufacturer. If the manufacturer of the reagents or instruments used is not indicated, they are all conventional products that can be purchased commercially.

### Embodiment 1

This embodiment provides a hydroformylation reaction method for catalyzing propylene using a catalyst, which includes the following steps:

### (1) Preparation of Iridium-Based Catalysts

Under the protection of argon atmosphere, 100 ml of tetrahydrofuran, 5 g of iridium chloride cyclooctadiene, 7.9 g of tripyridyl-phosphine are added into a 250 ml three-mouth flask, stirring at 5° C. for 2 h, and then tetrahydrofuran is removed at 30° C. under vacuum conditions. Thus, 10.5 g of tripyridyl-phosphine iridium-based catalyst can be obtained, which is an iridium-based catalyst.

### (2) Hydroformylation Reaction

Adding 9 mg of iridium-based catalyst prepared in step (1) and 5 ml of acetonitrile into the 50 ml high-pressure reactor, after hydrogen gas is introduced to replace the gas three times, 5 bar propylene, 8 bar carbon monoxide and 8 bar hydrogen are introduced in turn, the temperature is raised to 100° C. under stirring, the reaction is 8 hours, and after the reaction is completed, the reaction solution is cooled to 0° C., the pressure is slowly relieved, and samples are taken for gas chromatography detection.

The detection method is as follows: the reaction solution is sampled using the meteorological chromatography internal standard method to calculate the production yields of n-butyraldehyde and isobutyraldehyde. The ratio of n-butyraldehyde to isobutyraldehyde is the ratio of the production yields of n-butyraldehyde and isobutyraldehyde. TON is the total production yield (molar amount) of butyraldehyde compared to the input amount of catalyst (molar amount).

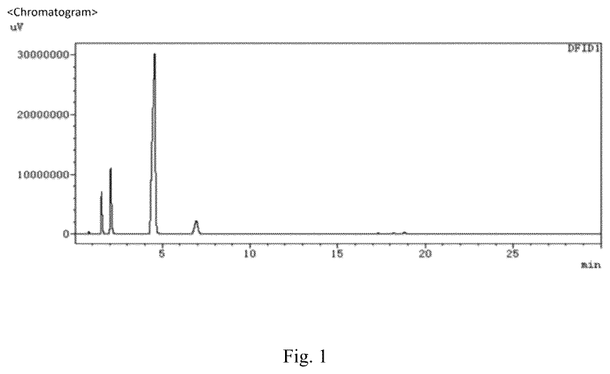

The gas chromatogram is shown in FIG. 1. The detection results are: the selectivity of n-butyraldehyde and isobutyraldehyde is 99.5%, the TON is 310, and the ratio of n-butyraldehyde to isobutyraldehyde is 1.7.

### Embodiment 2

The specific operation steps are consistent with Embodiment 1, except that the adopted iridium-based catalyst is different. The iridium-based catalyst adopted in Embodiment 2 is a tripyridyl-phosphine-tetrafluoroborate iridium-based catalyst. The preparation method includes the following steps:

Under the protection of nitrogen atmosphere, adding 100 ml of tetrahydrofuran and 3.2 g of silver tetrafluoroborate into a 250 ml three-mouth flask, the reaction is carried out at room temperature for 6 h in the dark, and removing the insoluble matter by filtration, and then adding 7.9 g of tripyridyl-phosphine, stirring at 5° C. for 2 h, and then removing tetrahydrofuran at 30° C. under vacuum conditions. Thus, 11.0 g of tripyridyl-phosphine-tetrafluoroborate iridium-based catalyst can be obtained, which is an iridium-based catalyst.

A catalytic reaction is conducted by adopting the prepared iridium-based catalyst above, and the gas chromatography results show that: the selectivity of n-butyraldehyde and isobutyraldehyde is 99.5%, the TON is 310, and the ratio of n-butyraldehyde to isobutyraldehyde is 1.6.

### Embodiment 3

The specific operation steps are consistent with Embodiment 1, except that the adopted iridium-based catalyst is different. The iridium-based catalyst adopted in Embodiment 3 is a dipyridyl-phenyl-phenyl-phosphine iridium-based catalyst. The preparation includes the following steps:

Under the protection of nitrogen atmosphere, adding 100 ml of tetrahydrofuran, 5 g of iridium chloride cyclooctadiene, and 7.9 g of dipyridyl-phenyl-phosphine phosphine into a 250 ml three-mouth flask, stirring at 5° C. for 2 h, and then removing tetrahydrofuran at 30° C. under vacuum conditions. Thus, 10.6 g of dipyridyl-phenyl-phenyl-phosphine iridium-based catalyst can be obtained, which is an iridium-based catalyst.

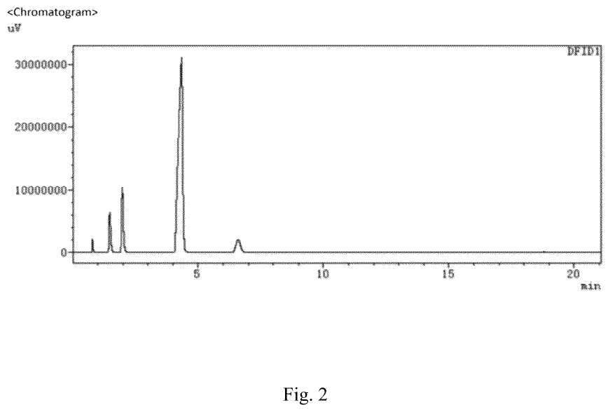

A catalytic reaction is conducted by adopting the prepared iridium-based catalyst above, and the gas chromatography is showed as FIG. 2. The detection results show that: the selectivity of n-butyraldehyde and isobutyraldehyde is 99.7%, the TON is 306, and the ratio of n-butyraldehyde to isobutyraldehyde is 1.6.

### Embodiment 4

The specific operation steps are consistent with Embodiment 1, except that the adopted iridium-based catalyst is different. The iridium-based catalyst adopted in Embodiment 4 is a pyridyl-diphenyl-phosphine iridium-based catalyst. The preparation includes the following steps:

Under the protection of argon atmosphere, adding 100 ml of tetrahydrofuran, 5 g of iridium chloride cyclooctadiene, and 7.8 g of pyridyl-diphenyl-phosphine into a 250 ml three-mouth flask, stirring at 5° C. for 2 h, and then removing tetrahydrofuran at 30° C. under vacuum conditions. Thus, 10.4 g of pyridyl-diphenyl-phosphine iridium-based catalyst can be obtained, which is an iridium-based catalyst.

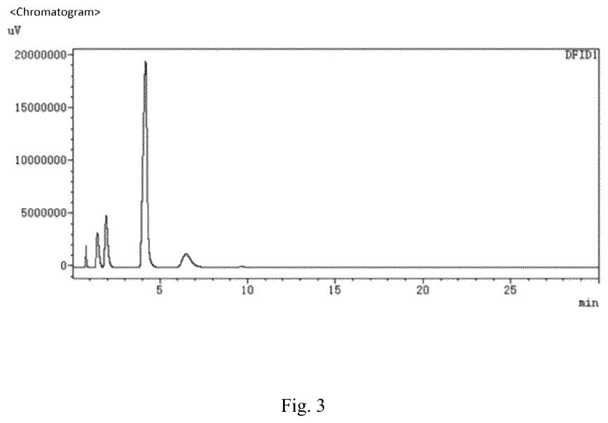

A catalytic reaction is conducted by adopting the prepared iridium-based catalyst above, and the gas chromatography is showed as FIG. 3. The detection results show that: the selectivity of n-butyraldehyde and isobutyraldehyde is 99.6%, the TON is 297, and the ratio of n-butyraldehyde to isobutyraldehyde is 1.8.

### Embodiment 5

The specific operation steps are consistent with Embodiment 1, except that the adopted iridium-based catalyst is different. The iridium-based catalyst adopted in Embodiment 5 is a tripyridyl-phosphine-iridium nitrate catalyst. The preparation method includes the following steps:

Adding 5 g of iridium chloride cyclooctadiene, 2.8 g of silver nitrate, and 100 ml of tetrahydrofuran into a 250 ml three-mouth flask, the reaction is carried out at room temperature for 6 h in the dark, and removing the insoluble matter by filtration, and then adding 7.9 g of tripyridyl-phosphine, and the remaining operation steps are the same as Embodiment 1. Thus, 9.8 g of tripyridyl-phosphine-iridium nitrate catalyst can be obtained, which is an iridium-based catalyst.

A catalytic reaction is conducted by adopting the prepared iridium-based catalyst above, and the gas chromatography results show that: the selectivity of n-butyraldehyde and isobutyraldehyde is 99.7%, the TON is 308, and the ratio of n-butyraldehyde to isobutyraldehyde is 1.7.

### Embodiments 6-8

The specific operation steps are consistent with Embodiment 1, except that the reaction conditions in Step (1) are changed. The specific circumstances are as shown in Table 1 below:

TABLE 1
Impacts of reaction conditions in Step (1) on experimental results.

| | Dropwise | | Vacuum | Selectivity of | Turnover | |
|---|---|---|---|---|---|---|
| | Addition | Stirring | Evaporation | n-butyraldehyde | number of | |
| | Temperature | Temperature | Temperature | and | Catalyst | n-butyraldehyde/ |
| Embodiments | (° C.) | (° C.) | (° C.) | isobutyraldehyde | (TON) | isobutyraldehyde |
| 6 | 0 | 40 | 25 | 99.6% | 261 | 1.7 |
| 7 | 30 | 20 | 50 | 99.5% | 210 | 1.6 |
| 8 | 15 | 35 | 40 | 99.5% | 243 | 1.6 |

### Embodiments 9-18

The specific operation steps are consistent with Embodiment 1, except that the reaction conditions in Step (2) are changed. The specific circumstances are as shown in Table 2 and Table 3 below:

TABLE 2
Impacts of reaction temperatures on experimental results.

| | Reaction | Selectivity of | Turnover | |
|---|---|---|---|---|
| | Temper- | n-butyraldehyde | number of | |
| Embodi- | ature | and | Catalyst | n-butyraldehyde/ |
| ments | (° C.) | isobutyraldehyde | (TON) | isobutyraldehyde |
| 9 | 80 | 99.8% | 137 | 2.4 |
| 10 | 90 | 99.7% | 233 | 1.9 |
| 11 | 100 | 99.5% | 310 | 1.7 |
| 12 | 110 | 99.6% | 336 | 1.4 |
| 13 | 120 | 99.4% | 299 | 1.1 |

TABLE 3
Impacts of partial pressure ratios of carbon monoxide to hydrogen on experimental results.

| | | | | Selectivity of | | |
|---|---|---|---|---|---|---|
| | | Carbon | | n-butyraldehyde | Turnover number | |
| | Propylene | monoxide | Hydrogen | and | of Catalyst | n-butyraldehyde/ |
| Embodiments | (bar) | (bar) | (bar) | isobutyraldehyde | (TON) | isobutyraldehyde |
| 14 | 5 | 15 | 3 | 99.5% | 335 | 2.3 |
| 15 | 5 | 12 | 6 | 99.7% | 316 | 1.9 |
| 16 | 5 | 9 | 9 | 99.6% | 349 | 1.7 |
| 17 | 5 | 6 | 12 | 99.5% | 246 | 1.3 |
| 18 | 5 | 3 | 15 | 99.3% | 210 | 1.1 |

### Embodiments 19-24

The specific operation steps are consistent with Embodiment 1, except that the choices of solvents in Step (2) are changed. The specific circumstances are as shown in Table 4 below:

TABLE 4
Impacts of different solvents on experimental results.

| | | Selectivity of | Turnover | n-butyral- |
|---|---|---|---|---|
| | | n-butyraldehyde | number of | dehyde/ |
| Embodi- | | and | Catalyst | isobutyral- |
| ments | Solvent | isobutyraldehyde | (TON) | dehyde |
| 19 | Toluene | 99.6% | 99 | 1.4 |
| 20 | N-methyl- | 99.6% | 283 | 1.7 |
| | pyrrolidone | | | |
| 21 | 1,4-dioxane | 99.4% | 297 | 1.4 |
| 22 | N,N-dimethyl- | 99.5% | 340 | 1.5 |
| | formamide | | | |
| 23 | isobutyral- | 99.7% | 284 | 3.1 |
| | dehyde | | | |
| 24 | n-butyral- | 99.8% | 276 | 0.5 |
| | dehyde | | | |

### Embodiments 25-29

The specific operation steps are consistent with Embodiment 1, except that the mass ratios of the concentration of the catalyst relative to the solvent are changed. The specific circumstances are as shown in Table 5 below:

TABLE 5
Impacts of concentration of catalysts on experimental results.

| | Mass Ratio of | | | |
|---|---|---|---|---|
| | Concentration of | Selectivity of | Turnover | n-butyral- |
| | Catalyst relative | n-butyraldehyde | number of | dehyde/ |
| Embodi- | to Solvent | and | Catalyst | isobutyral- |
| ments | (wt %) | isobutyraldehyde | (TON) | dehyde |
| 25 | 0.01 | 99.5% | 524 | 1.6 |
| 26 | 1.0 | 99.6% | 40 | 1.7 |
| 27 | 0.3 | 99.5% | 245 | 1.7 |
| 28 | 0.6 | 99.7% | 133 | 1.7 |
| 29 | 0.04 | 99.6% | 510 | 1.6 |
| 30 | 1.5 | 99.5% | 27 | 1.7 |

### Comparative Example 1

The specific implementation manner is consistent with Embodiment 1, the only difference is that the vacuum evaporation temperature is selected as 50° C., and the produced iridium-based catalyst above is used for performing the catalytic reaction. The detection results of gas chromatography are: the selectivity of n-butyraldehyde and isobutyraldehyde is 99.6%, the TON is 228, and the ratio of n-butyraldehyde to isobutyraldehyde is 1.6.

As can be seen from the experimental data of Comparative Example 1 that: when the vacuum evaporation temperature exceeds the limit range of the present invention, the effect of the produced catalyst in Comparative Example 1 is far lower than the effect of the catalyst in Embodiment 1. This is because only within the temperature range of vacuum evaporation limited by the present invention can the phosphine ligand be prevented to be oxidized during the reaction process and ensure that the phosphine ligand and the metal iridium can play a good catalytic effect.

### Experimental Example 1

Tripyridyl-phosphine iridium-based catalyst and tripyridyl-phosphine rhodium-based catalyst are respectively used as catalysts. The same molar amounts of catalysts are added to conduct the reaction under the conditions described in Embodiment 1, and reaction results are shown in Table 6 below:

TABLE 6
Comparison of reaction results.

| | Selectivity of | Turnover | |
|---|---|---|---|
| | n-butyraldehyde | number of | |
| | and | Catalyst | n-butyraldehyde/ |
| Catalyst | isobutyraldehyde | (TON) | isobutyraldehyde |
| Rhodium-based | 99.6% | 190 | 1.4 |
| Catalyst | | | |
| Iridium-based | 99.7% | 310 | 1.7 |
| Catalyst | | | |

As can be seen from the reaction results in Table 6 above that, the catalytic activity of the tripyridyl-phosphine iridium-based catalyst is better than that of the tripyridyl-phosphine rhodium-based catalyst under the condition that the same ligand (tripyridyl-phosphine) is used. This is because the pyridine group in the tripyridyl phosphine used in the present invention is an electron-deficient group relative to other ligands. When the catalytic reaction is conducted, the pyridine group has low activation ability for metal rhodium, but the pyridine group in the tripyridyl phosphine has good activation performance for metal iridium. When the pyridine group in the tripyridyl phosphine is not substituted, the catalytic performance of the produced tripyridyl-phosphine iridium-based catalyst is the highest. At the same time, it can also be seen from the experimental data in Table 6 above that: the turnover number (TON) of rhodium-based catalyst is much lower than that of iridium-based catalyst under the condition that the ligand is tripyridyl phosphine, which further proves that the activity ability of tripyridyl group relative to rhodium metal is much lower than that of iridium metal.

During the specific using process, the iridium-based catalyst prepared by the present invention can realize the hydroformylation of propylene under mild conditions, and the production cost can be greatly reduced in production. Moreover, the iridium-based catalyst can still maintain good catalytic activity during recycling process.

### Experimental Example 2

The catalyst after the reaction in Embodiment 1 is detected, and then the solvent is removed at 25° C. under vacuum evaporation, and the resulting catalyst is re-added into a fresh solvent for performing recycle and reuse of catalyst, and the results are shown in Table 7 below:

TABLE 7
Experimental results of recycle and reuse of catalysts.

| | Selectivity of | Turnover | |
|---|---|---|---|
| | n-butyraldehyde | number of | |
| Number of | and | Catalyst | n-butyraldehyde/ |
| cycles | isobutyraldehyde | (TON) | isobutyraldehyde |
| 1 | 99.6% | 310 | 1.6 |
| 2 | 99.7% | 300 | 1.7 |
| 3 | 99.6% | 302 | 1.5 |
| 4 | 99.6% | 299 | 1.6 |
| 5 | 99.5% | 289 | 1.5 |

It can be seen through the above-mentioned experimental data that, the catalyst prepared by the present invention can still maintain a good catalytic effect after being used many times. It also shows that the catalyst disclosed in the present invention can still participate in the production after being used many times, and can greatly reduce the expenditure on the catalyst, and save resources.

Finally, it should be noted that the above embodiments are merely used to illustrate the technical schemes of the present invention, rather than to limit the present invention. Although the present invention has been described in detail with reference to the above-mentioned embodiments, those of ordinary skill in the art should understand that they can still modify the technical schemes recorded in the above-mentioned embodiments or make equivalent substitutions for some or all of the technical features. However, these modifications or substitutions do not cause the essence of the corresponding technical scheme to depart from the scope of the technical scheme of each embodiment of the present invention.

What is claimed is:

1. An iridium-based catalyst, wherein a structural formula of the iridium-based catalyst is: 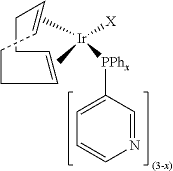
wherein, Ph is a phenyl group, x is any one of 0, 1, 2, and X is any one of Cl, NO₃, BF₄, PF₆ and acac.

2. A preparation method of the iridium-based catalyst of claim 1, comprising the following steps:
under the protection of inert gas, adding dropwise a tetrahydrofuran solution of phosphine ligand to a tetrahydrofuran solution of iridium compound, stirring after dropping, and vacuum evaporating the tetrahydrofuran solution at 25-30° C. to obtain a target product.

3. The preparation method of claim 2, wherein a temperature of dropwise addition is 0-30° C., a temperature of stirring is 20-40° C., and the inert gas is one of argon or nitrogen;
wherein the temperature of dropwise addition is 5° C., the temperature of stirring is 30° C., and the temperature of vacuum evaporation is 30° C.

4. The preparation method of claim 2, wherein a molar ratio of the iridium compound to the phosphine ligand is 1:2, and the iridium compound is iridium chloride cyclooctadiene.

5. The preparation method of claim 2, wherein the phosphine ligand is any one or more of tripyridyl phosphine, dipyridyl-phenyl-phenyl-phosphine, and pyridyl-diphenyl-phosphine.

6. A hydroformylation reaction method for catalyzing propylene using a catalyst, comprising the following steps:
adding the catalyst and a corresponding solvent, dispersing evenly, and adding propylene, carbon monoxide and hydrogen for reaction;
wherein the catalyst is selected from the iridium-based catalyst of claim 1 or the iridium-based catalyst prepared by any one of claims 2-5;
wherein the catalyst accounts for 0.01-1.0 wt % of a solvent mass;
wherein the catalyst accounts for 0.2 wt % of the solvent mass.

7. The method of claim 6, wherein a partial pressure ratio of the carbon monoxide to the hydrogen is 1:(0.2-5);
wherein the partial pressure ratio of the carbon monoxide to the hydrogen is 1:1.

8. The method of claim 6, wherein the solvent is a combination of any one or more of n-butyraldehyde, isobutyraldehyde, toluene, acetonitrile, N-methylpyrrolidone, 1,4-dioxane, N,N-dimethylformamide;
wherein the solvent is acetonitrile.

9. The method of claim 6, wherein a temperature of the reaction is 80-120° C., and a pressure of the reaction is 1.5-3.8 MPa;
wherein the temperature of the reaction is 100° C., and the pressure of the reaction is 1.9 MPa.

10. The method of claim 6, wherein an addition of iridium metal in the iridium-based catalyst relative to the solvent is 0.003-0.3 wt %;
wherein the addition of iridium metal in the iridium-based catalyst relative to the solvent is 0.07 wt %.
